(19) United States
(12) Patent Application Publication
(10) Pub. No.: US 2010/0275928 A1
(43) Pub. Date: Nov. 4, 2010

(54) Professional massage draping sheet set

(76) Inventor: Esther Marianne Roberts, Las Vegas, NV (US)

Correspondence Address: Esther Marianne Roberts, 6204 Old Trail Road, Las Vegas, NV, 89108, US

(21) Appl. No.: 12/799,333

(22) Filed: Apr. 22, 2010

Related U.S. Application Data
(60) Provisional application No. 61/202,952, filed on Apr. 22, 2009.

(51) Int. Cl. A61B 19/08

(52) U.S. Cl. 128/849

(57) ABSTRACT

A professional massage draping sheet set comprised of a one piece textile fabric featuring a veil covered abdomen opening to access the stomach region and two openings for each leg with an angle cut to the pelvic region. All openings are fastened with special open-closure mechanism including light-weight, strong-hold magnets. And a second professional massage fitted sheet fitted for professional massage tables that includes an opening at each end to accommodate access to the head and foot rest attachments.

### CROSS-REFERENCE TO RELATED APPLICATIONS

The present application claims the benefit of Provisional Patent Application No. 61/202,952 filed on Apr. 22, 2009 by the present inventor.

### STATEMENT REGARDING FEDERALLY SPONSORED RESEARCH OR DEVELOPMENT

NOT APPLICABLE

### REFERENCE TO SEQUENCE LISTING, A TABLE, OR A COMPUTER PROGRAM LISTING COMPACT DISC APPENDIX

NOT APPLICABLE

### FIELD OF THE INVENTION

This invention relates to a draping sheet set, specifically which are used for working with the human body's muscular system requiring legal draping coverage yet necessary access to certain body parts.

### BACKGROUND OF THE INVENTION

Historically, a conventional bed sheet and fitted sheet are used to accommodate the profession of massage therapy during a massage. These conventional bed sheet set coverings are limited as to the required moveably of fabric to access particular body parts for massaging without exposing gentile areas. Such conventional bed sheets are used to cover the client's body to avoid nudity, provide protection and warmth.

The conventional twin size format is used to accommodate the professional massage table as this is the size that fit closest to the table. The client undresses before laying down either prone or supine on the table, and then pulling the sheet over the body.

The massage therapist then manipulates the bed sheet with acquired professional massage draping procedures and the help of the client holding such bed sheet to access body parts like the abdomen or legs without exposing gentile areas. One of the major downsides of bed sheets used for massage draping is the inability to access the client's stomach area without having to move the sheet away from the breast tissue area. Traditionally, and legally required, a massage therapist must use another piece of fabric such as a pillow case, then placing the pillow case over the bed sheet where the client's breast tissue is assumed to be and then carefully removing the bed sheet from underneath while the client hold the pillow case in place. This procedure is extremely vulnerable to exposure leaves the client fully exposed with only a pillow case coverage and remains cumbersome and uncomfortable to the client.

Furthermore, the leg drape is an acquired draping procedure and almost always requires finesse and experience by the therapist. The traditional leg drape is very depended to the bed sheet due to the limitations of its design features of a singular rectangular sheet. The therapist has to grab the lateral side of the bed sheet residing on top of the client and then moving it between the crutch areas. The client's leg is then slightly lifted and held up while the therapist grabs the sheet from underneath and bringing it from the middle crutch area to the lateral side. The sheet is then wrapped with an upward pulling motion to secure the sheet. The client is then asked to secure the sheet by holding onto it while the leg is being massaged. When the therapist is done with the leg massage, the therapist returns the sheet back in reverse by lifting the leg, pulling the sheet from underneath and returning it back to the top of the client. Because the bed sheet is only a one-piece fabric sheet, it is extremely difficult to manipulate the sheet to accommodate the anatomy of the human body where the sheet has to fulfill coverage, necessary exposure, and mandatory drape all in one. Additionally, these cumbersome draping procedures cause the flow of the massage to be interrupted each time a body part is accessed. For centuries we have accepted a conventional bed sheet as the single solution for massaging. While legal draping requirements for massaging have changed over the last few years as a necessary part of this profession, a sheet that can accommodate such requirements is warranted rather than trying to continue to manipulate a limited one piece bed sheet. Not only will it show that the present invention is a massage sheet for client coverage but also a tool for massage therapists obligated to conform to these requirements reducing the potential for exposure and increasing the comfort level combined.

Thereafter, inventors have tried to invent drape sheets that focus mainly on the coverage of the client. In some parts make the sheet more movable yet it still appears that none of the inventions actually address the need of a massage therapist's necessary body part accessibility. This is needed to massage the human body, maintain the flow of the massage without having to perform major sheet manipulation, and at the same time create the needed legal coverage stated by law all in one application. Inventors created several types of drape sheets for the massage or medical industry U.S. Pat. No. 7,496,971 to Soto (2009) shows a drape sheet that is folded in half with hook and loop fasteners which are cumbersome and generally focus on the complete coverage of the client versus accessibility and coverage while maintaining the flow of the massage. This said invention also excludes the accessibility to the abdomen which is an important aspect of the massage modalities. Another invention for a drape sheet U.S. Pat. No. 5,274,852 to Hogan (1994) requires the client to encompass the head through an open seam garment more applicable for medical examinations than the massage therapy. Additionally, this garment is more of a one-piece suit rather than a flat sheet drape.

Additionally, the fitted sheet used for massage therapy is generally a standard twin size fitted bed sheet with a rubber band design to hold the sheet snug on the table. Every massage table has two attachment locations at either end of the table to insert a head or foot rest attachment which is used for every massage. Because of the fitted sheet's design, the sheet will cover up these openings making them inaccessible to the therapist. To access them, the therapist has to hold the head or foot rest cover in one hand, move and hold the fitted sheet up to expose the attachment point and at the same time try to insert the head or foot rest device into the two holes. Not only is this a cumbersome process, it could also damage the sheet or cause the sheet to get tangled up.

Because of these limitations to access required body parts, provide coverage and warmth, maintain legal draping of sensitive gentile areas and maintain the flow of the massage all at the same time, the need to re-design a sheet is warranted. Accordingly, I invented the professional massage drape sheet set of which several objects and advantages of the present invention are:

(a) to provide access to the abdomen through an opening that is covered by a veil with quiet magnets; no pillow case required

(b) to provide access to each leg and pelvis region individually through an opening that is provided just for these specific regions;

(c) to provide a fastening device for the openings through light-weight strong-hold magnet attachments which are quiet, easy to separate, and quickly to fasten without interrupting the flow of the massage;

(d) a sheet that is designed in such a way which does not need lengthy manipulation by the therapist to accomplish accessibility while maintaining legal coverage;

(e) a sheet that accommodates the necessary massage modalities and at the same time addresses the legal draping requirements when providing a massage without having to use cumbersome draping procedures.

(f) a sheet designed in such a way where a one-piece flat sheet turns into a three piece moveable sheet for accessibility in the leg and pelvic region and coverage protection in the crutch area from the middle section piece.

(g) a sheet that is made from natural fiber material suitable for the massage therapy and comfort of the client and which is large enough to fit diverse body types

(h) a fitted sheet that is designed to fit the standard massage table size allowing enough extra fabric to insert a massage bolster and an opening on each end designed to insert a foot or head rest device.

The invention reflects the above referenced solutions for most massage therapy modalities using a single utility design of the present invention to be used as a tool. Moreover, the present invention also incorporates a fastening system that is fitting for the massage therapies healing aspects and need for low noise by introducing magnets as a fastening mechanism. Combining the re-design of the sheet as a tool, and using the preferred method of magnet closures for a fastening system, coupled with the therapist professional massage application promotes healing, comfort, and professional handling of the sheet all in one device.

Therefore, the present invention reflects the re-design of the massage drape sheet and the fitted sheet as a professional massage drape sheet set.

### BRIEF SUMMARY OF THE INVENTION

In accordance with the present invention a massage drape sheet having an abdomen opening that is covered by a veil, a right and a left leg opening angled to the pelvic region and all said openings fastened with light weight, strong hold magnets. And a massage fitted sheet with two openings, one at each end to accommodate the insertion of a head or foot rest attachment.

These and other objects and advantages of the present invention will become apparent following the further review of the drawings and specifications of the invention.

### BRIEF DESCRIPTION OF THE SEVERAL VIEWS OF THE DRAWINGS

FIG. 1 is a top view of a massage drape sheet in accordance with the present invention illustrating the positions of an abdomen veil attachment and a right and left leg and pelvic opening in a fastened, closed position and placement of magnets.

FIG. 2 is a top view of the massage drape sheet showing the abdomen opening cutting lines, the leg and pelvic cutting lines, the veil attachment line, and the placements of the fabric patches for magnet attachments.

FIG. 3A, FIG. 3B, FIG. 3C, and FIG. 3D are detail views of a top pelvic magnet border attachment and a bottom pelvic magnet border attachment illustrating the magnet positions for each, the right and left leg respectively.

FIG. 4A, FIG. 4B, FIG. 4C and FIG. 4D are detail views of a top leg magnet border attachment and a bottom leg magnet border attachment illustrating the magnet positions for each, the right and left leg respectively.

FIG. 5 is a partial exploded view of FIG. 3A joined with FIG. 4A and FIG. 3B joined with FIG. 4B.

FIG. 6 is a partial detail view of the leg and pelvic magnet border attachments to the massage drape sheet's leg and pelvic cutting lines in accordance with the present invention.

FIG. 7 is a detail view of the fabric patch element to attach a magnet to the massage drape sheet.

FIG. 8 is a partial detail view of the abdomen veil's magnet border illustrating the placement and stitch lines for magnets.

FIG. 9 is a top view of a massage fitted sheet in accordance with the present invention illustrating the cutting lines and positions of an opening intended for a foot and/or head rest device.

### DRAWINGS

### Reference Numerals

01 massage drape sheet
09 massage fitted sheet
21 abdomen cutting line
22 abdomen opening
23 abdomen veil attachment
24 stitch line for abdomen veil attachment
25 abdomen veil magnet border
32 cutting line for leg/pelvic opening
34 stitch line for pelvic to leg connection
35 border attachment extension
36a right pelvic magnet border attachment top
36b right leg magnet border attachment top
36c right pelvic magnet border attachment bottom
36d right leg magnet border attachment bottom
37a left pelvic magnet border attachment top
37b left leg magnet border attachment top
37c left pelvic magnet border attachment bottom
37d left leg magnet border attachment bottom
40p magnets with positive side up
40n magnets with negative side up
41 stitch line to create magnet chamber pockets
42 fabric patch for magnet attachment
43 stitch line for fabric patch
51 cutting line for head/foot rest device opening
52 opening for head/foot rest device
60 right side of top view massage drape sheet
70 left side of top view massage drape sheet

### DETAILED DESCRIPTION OF THE DRAWINGS

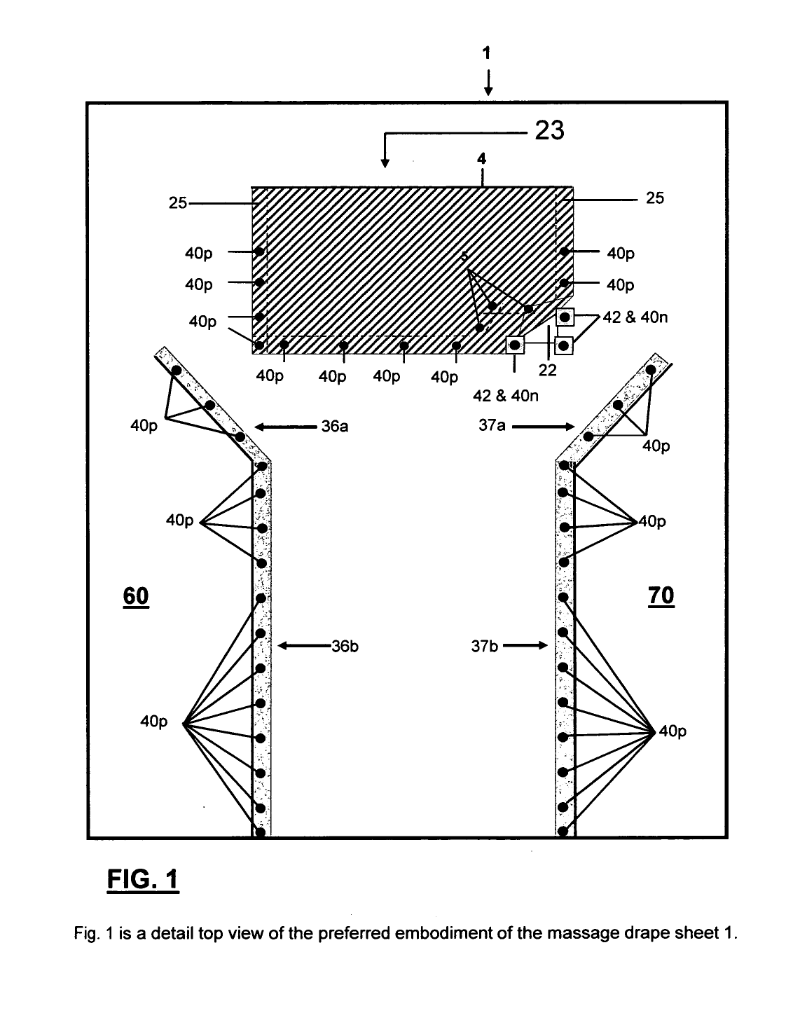

### FIG. 1—Preferred Embodiment

A preferred embodiment of the massage drape sheet 1 of the present invention is illustrated in FIG. 1 (front view). The massage drape sheet has an approximately overall size of 96 inches in length, 76 inches in width. The massage drape sheet has an approximate 1 inch hem border on the left, right, and bottom side and an approximate 3 inch hem border on the top side (not shown). As other dimensions may be employed it has been proven that the dimensions in the preferred embodiment are sufficient for the required draping coverage for all body types. As the sheet may be manufactured from sheet like material suitable for the massages of the required thread count, the preferred material is made from cotton like material or a blend of cotton thereof.

The massage drape sheet's 1 top view has two sides, a right side 60 and a left side 70 where the right leg/pelvic openings and respectively the left leg/pelvic openings are located. The massage drape 1 has five major elements comprised of the abdomen opening 22 (partially shown), the abdomen veil 23, and two leg/pelvic openings 32 (actual opening not shown), one for the right leg and one for the left leg. The fifth major element is the closure mechanism for each opening comprised of a magnetic attraction force pulling two separate fabric pieced together through the said magnetic force. The strength of the selected magnet is powerful enough to keep the fabric pieces in a fastened position during movement by the client, yet small, light-weight and manageable to separate by human force. Once separated, the fabric becomes the dominate force keeping the opening in an open position without the magnets interfering. It isn't until the therapist lines up the magnets located inside special made borders when the magnetic force is re-activated for the closure mechanism.

In the preferred embodiment, each magnet is approximately 6 mm in dia. (round shape) and approximately 3 mm in thickness and made preferably from the material neodymium with nickel coating, strong hold, and at least withstanding 80° degree Celsius heat.

The abdomen veil 23 is a separate element made from the same material as the massage drape sheet 1 and is approximately 25 inches in width, and approximately 18 inches in length with an approximate 1 inch wide magnet veil border 25 along the left, right, and bottom of the abdomen veil 23. This magnet veil border 25 has about 13 magnets 40p with the positive magnetic side facing up inside said border. These magnets 40p are spaced evenly about 4 inches apart along the left, right, and bottom side of the veil with at least one magnet 40p positioned in each lower right and lower left corner. A stitched line 41 on the left and right side of each magnet 40p will create at separate chamber within the border as illustrated in FIG. 8 to avoid contact between the same magnets inside the veil border and to keep the magnets in place.

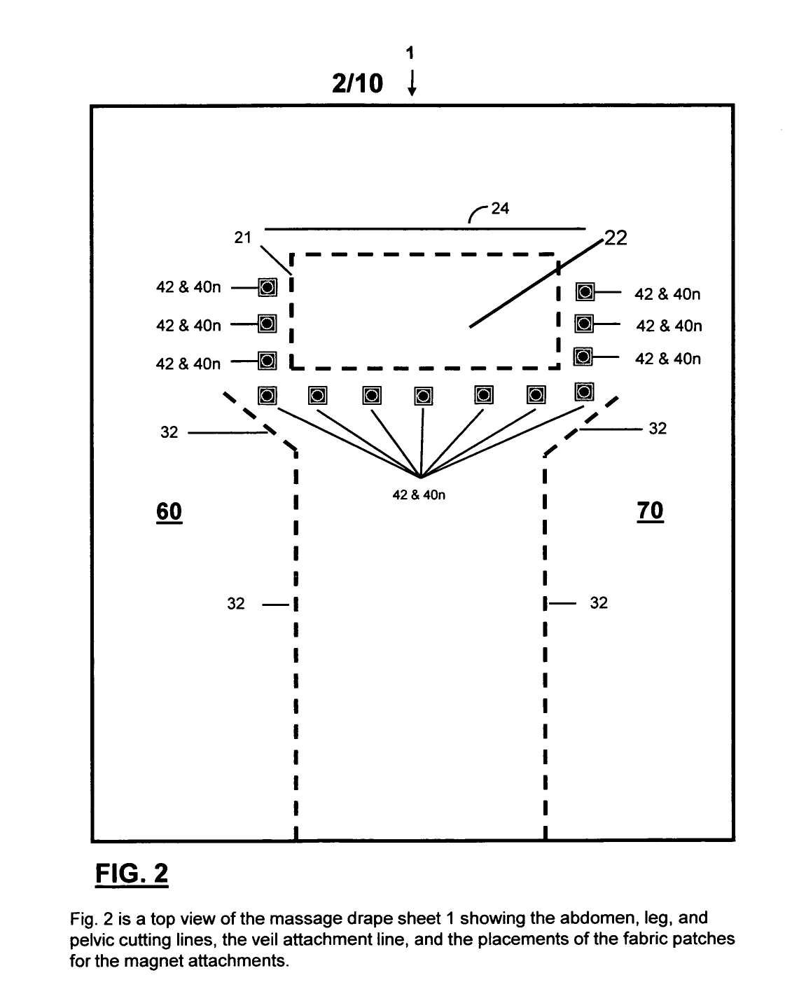

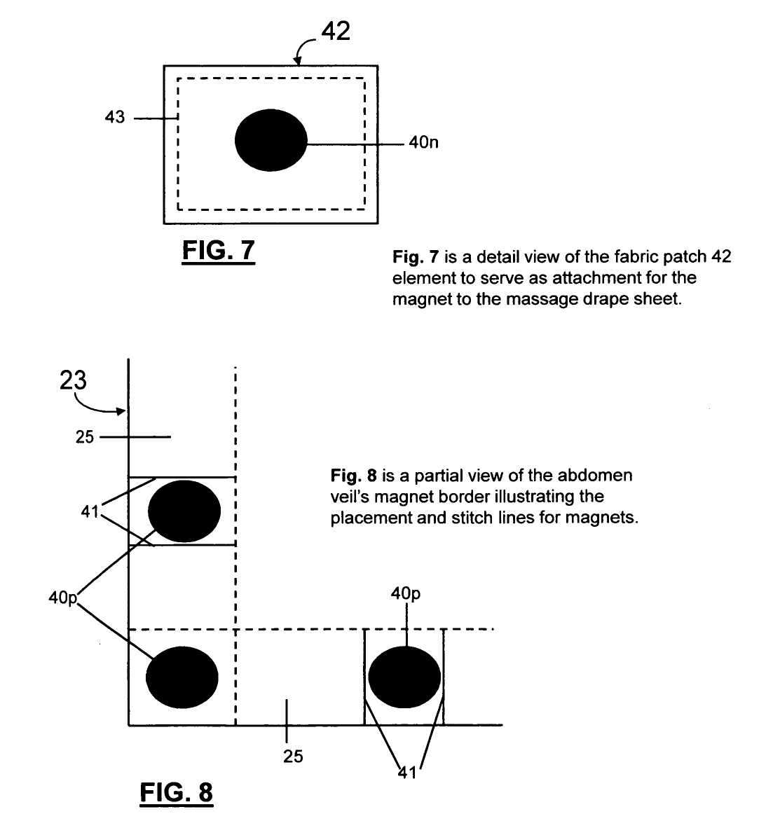

Accordingly, the opposite magnets 40n are sewn with a separate fabric patch 42 as illustrated in FIG. 7 directly onto the massage drape 1 as illustrated in FIG. 2. The closure mechanism is created by the magnets 40p inside the veil being attracted through the magnetic force from the magnets 40n mounted onto the massage drape sheet 1. The abdomen veil 23 element is placed about 14 inches from the top edge and about 27 inches from the left and right edge of the massage drape sheet 1 to completely cover the abdomen opening 22 (partially shown) and as illustrated in FIG. 1 in its entirety. The abdomen veil 23 is then sewn onto the massage drape sheet 1 at the top end as indicated with stitch line 24 and connected permanently to the massage drape sheet 1.

The amount of magnets needed for the abdomen veil's 23 open and closure mechanism will require about 26 magnets in total.

The cutting lines for the leg and pelvic opening are illustrated in FIG. 2. These cuts create an outer and inner attachment site for the borders. The top borders for the right and left leg/pelvic angle 36a, 36b, 37a, and 37b are attached to the outer cutting edge of the opening. The bottom borders for the right and left leg/pelvic angle (not shown) 36c, 36d, 37c and 37d are attached to the inner cutting edge of the opening.

The amount of magnets needed for the left and right legs/pelvis's open and closure mechanism will require about 56 magnets in total.

The abdomen veil attachment magnet border 25, the top and bottom leg attachment borders 36b, 36d, 37b, and 37d and the top and bottom pelvic attachment borders 36a, 36c, 37a, and 37c are an important aspect of the present invention as they provide the container to hold the magnets for the open and closing mechanism preferred for massage draping sheet 1.

### OPERATION

### FIG. 1

The massage drape sheet 1 has an abdomen opening 22 with an abdomen veil 23 and anatomically to the human body; two leg openings. The massage drape sheet's 1 preferred user is that of a skilled person in the field of massage therapy or similar fields requiring the coverage of the entire body and specific access to body parts being massaged or stretched. The massage drape 1 is positioned over the client (person being massaged) and adjusted in such a way where it fit's the client's anatomy according to the bony landmarks of the abdominal region, the leg region and the pelvic region of that specific client. Once the massage drape sheet 1 is correctly positioned, the therapist can return the abdomen veil 23 by separating the magnets 40p and 40p from each other using slight human force. While holding the abdomen veil 23, the therapist gently folds the veil back only exposing the abdomen region of the client to begin massaging the stomach area. Upon completion of the abdomen massage, the therapist returns the abdomen veil 23 by holding the corners of the veil, moving it towards the magnets 40n located on the massage drape sheet 1 in proximity to the opening. The magnets will then “catch” by holding the veil in such a way that the magnets connect with their respective opposite pole magnet.

To open the leg borders to expose each respective leg, the therapist takes the bottom edge of the leg opening and starts by separating the magnets holding each side of the magnet border using human force. The fabric like material will separate and expose the leg to be massaged. If desired, the left and right fabric pieces can now be used for additional draping around the leg. The middle piece section created by the two leg cuts remain in the crutch area of the client for coverage. Depending on the client's anatomy, the pelvic angle serves as an extended region to be massaged. Many modalities require reaching the glut area of a client, thus allowing for additional access to these areas. As the therapist separates the magnets starting at the bottom of the leg opening, the therapist continues to the desired point and either stops separating the magnets and/or continues up to the section desired. This will allow for complete accessibility to necessary muscular human body areas required by massage modalities. To close the leg opening, the therapists takes the open end point and goes in reverse order holding the magnet borders in a straight edge over each other. By magnetic force, the magnets will line up to reach the opposing poled magnet and close the opening instantaneously and quietly.

### DETAILED DESCRIPTION

### FIG. 2-FIG. 8

Referring to FIG. 2 showing the massage drape sheet 1 of the preferred embodiment of the present invention illustrating the cutting line 21 for the abdomen opening 22 whereas a rectangular piece is being cut out of the massage drape sheet 1. The size of the abdomen opening 22 is approximately 22 inches in width and approximately 12 inches in height with an approximate ½ inch hem border around the cutting edge. The abdomen opening 22 is about 16½ inches from the top edge border and about 26½ inches from the outer left edge border and 26½ inches from the outer right edge border, and about 67 inches from the bottom edge border of the massage drape sheet 1.

The stitch line 24 for the abdomen veil 23 is approximately 1 inch above the top edge of the abdomen opening 22 and approximately 25 inches or the same length of the abdomen veil 23.

The same amount of magnets used in the abdomen veil attachment border 25 are placed at the exact opposite location with a fabric patch 42 as illustrated in FIG. 7 are used for mounting the magnets 40n with the negative magnetic side facing up. Each magnet is sewn with the fabric patch 42 and magnet 40n directly onto the massage drape sheet 1. The positioning of the magnets with the fabric patch 42 are spaced evenly approximately 4 inches apart and approximately 2 inches around the abdomen opening 22. When the abdomen veil 23 is in the fastened closed position, the magnets 40p inside the abdomen veil magnet border 25 and the magnets 40n mounted with the fabric patch 42 will create an attractive force between the magnets and keep the veil 23 in a fastened position to cover the abdomen opening 22 entirely.

Description of the top side of the said massage draping sheet 1 illustrating the right and left leg/pelvic cutting lines 32 to provide access to each leg and to provide an attachment site for the magnet borders 36a, 36b, 36c, 36d and 37a, 37b, 37c, 37d respectively. The starting point for the right leg/pelvic cut line 32 is located approximately 27 inches or ⅓ from the right side of the massage drape sheet's right border edge. The cut line 32 is elongated approximately 56 inches in length and then slightly angled (approximately 90° degree angle) to the right for approximately 11 inches in length creating the pelvic angle. The end point of the cut line 32 prior to angling to the right must be in distance approximately 8 inches away from the bottom right edge of the abdomen opening 22. The end point of the right pelvic angle cut line 32 must be in distance approximately 7 inches away from the right bottom corner edge of the abdomen opening 22.

The starting point for the left leg/pelvic cut line 32 is located approximately 27 inches or ⅓ from the left side of the massage drape sheet's left border edge. The cut line 32 is elongated approximately 56 inches in length and then slightly angled (approximately 90° degree angle) to the left for approximately 11 inches in length creating the pelvic angle. The end point of the cut line 32 prior to angling to the right must be in distance approximately 8 inches away from the bottom left edge of the abdomen opening 22. The end point of the left pelvic angle cut line 32 must be in distance approximately 7 inches away from the left bottom corner edge of the abdomen opening 22.

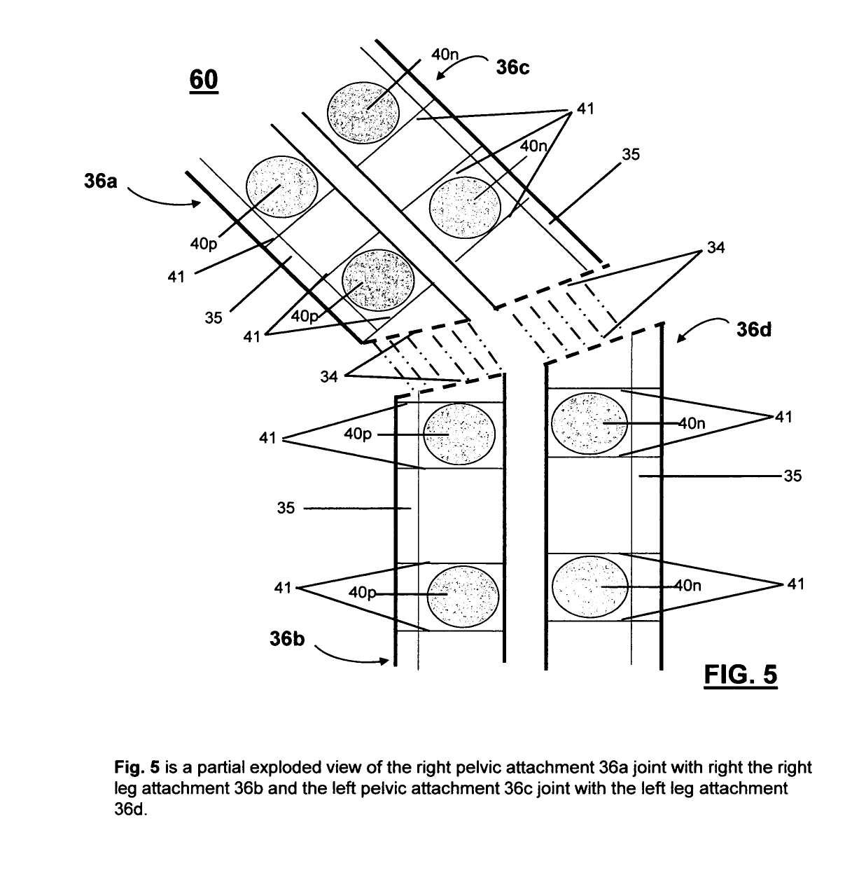

The opening created by the cutting line 32 also creates an outer side cutting edge and an inner side cutting edge for each respective left and right leg/pelvic opening. These cutting edges serve as the attachment sites for the magnet border attachments 36a, 36b, 36c, 36d and 37a, 37b, 37c, and 37d to create the open closure mechanism. The approximate ½ inch excessive fabric strip 35 as illustrated on FIG. 5 is used to attach each complete magnet border to each respective side of the cutting edge.

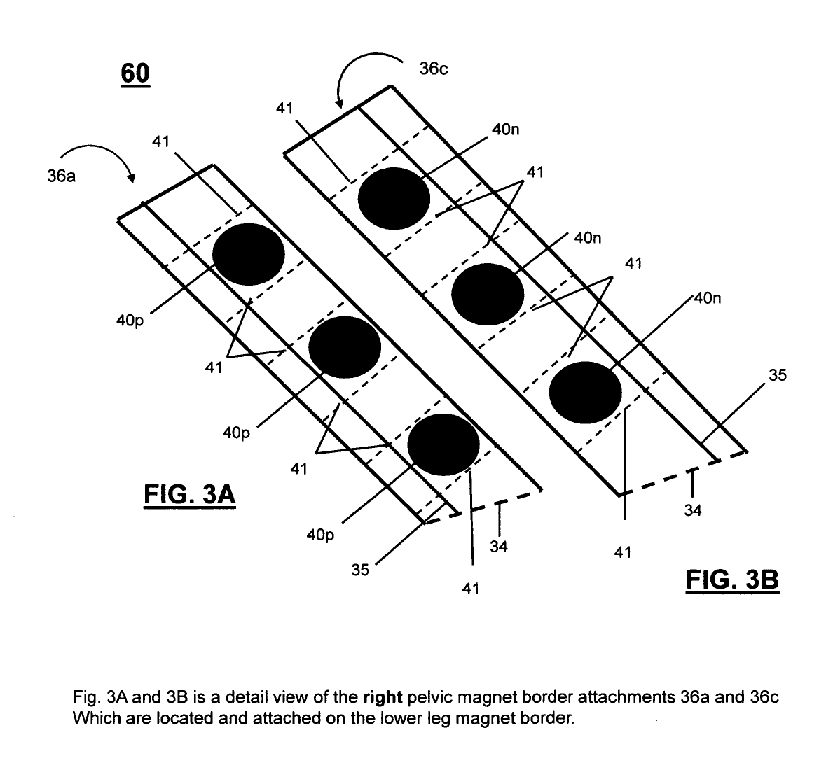

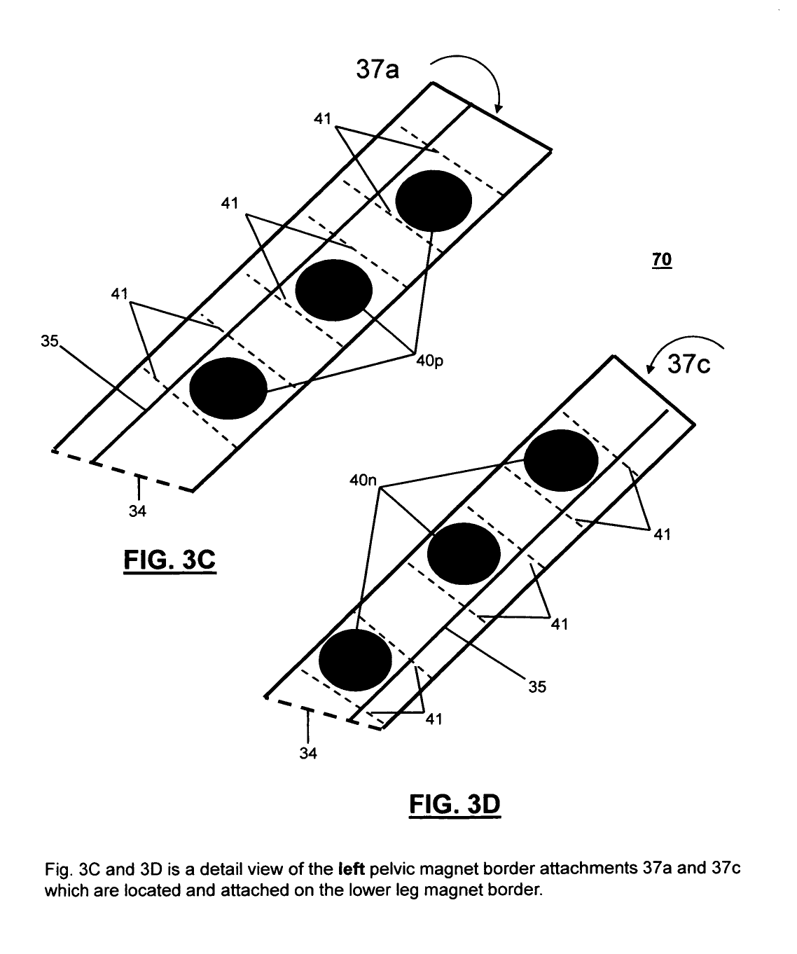

Referring to FIG. 3A through FIG. 3D are detail views of the pelvic magnet border attachments 36a, 36c and 37a, 37c respectively. Each said pelvic magnet borders 36a, 36c and 37a, 37c are made from the same material as the material of the massage drape sheet 1 and fabricated approximately 4½ inches in width and approximately 12 inches in length. The fabric is creased to reach a 2 inch width double layer by folding it over and an approximate ½ inch attachment sewing border 35 that will serve to attach the magnet borders to the massage drape sheet 1. The end of each border is cut and sewn to fit the leg/pelvic cut 32 of the massage drape sheet 1. About 3 magnets 40p in pelvic magnet borders 36a, 37a are spaced approximately 4 inches apart from the top to the bottom in between the top and bottom layer of the fabric. Respectively, about 3 magnets 40n in pelvic magnet border 36c, 37c are spaced approximately 4 inches apart from the top to the bottom in between the top and bottom layer of the fabric. Each magnet located inside the pelvic magnet border 36a, 36c and 37a, 37c respectively will then be sewn with a stitching line 41 on each side to create a chamber which keeps the magnet in place, in proper magnetic force position, and from moving within the border container. The entire border is then sewn along side together to close the border. The pelvic magnet border 36a, 36c and 37a, 37c are then attached to the leg magnet border 36b, 36d, 37b, 37c respectively at the sewing line 34 and as illustrated in FIG. 5 before being attached to each right/left; leg/pelvic cutting edge to the massage drape sheet 1.

In order to create a closing mechanism with magnets, the respective negative/positive magnetic side is positioned accurately prior to permanently sewing the borders onto the massage drape sheet 1. A chamber like pocket for each magnet is created by sewing two (a left and right) permanent stitch lines 41 where the magnet is located.

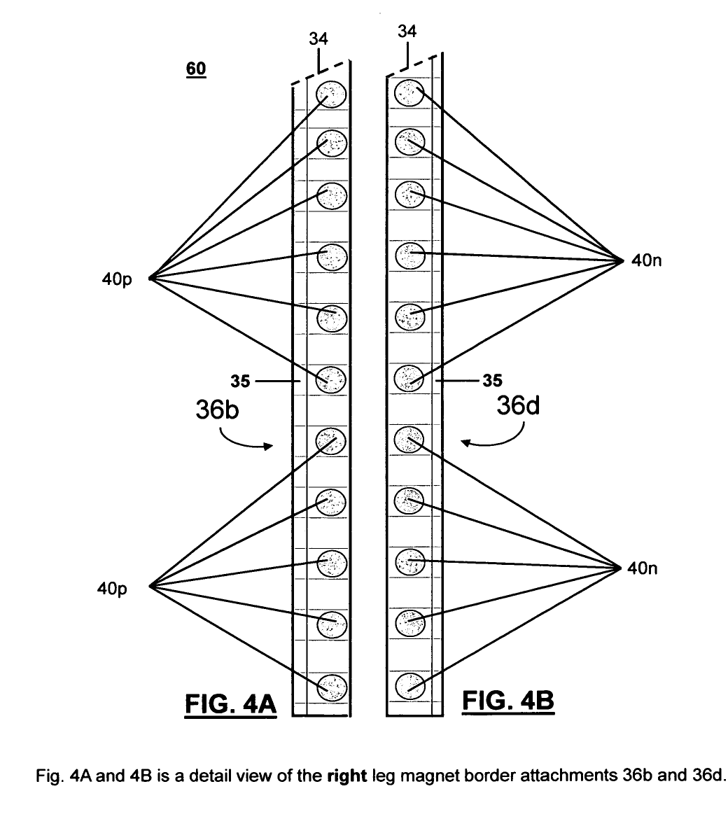

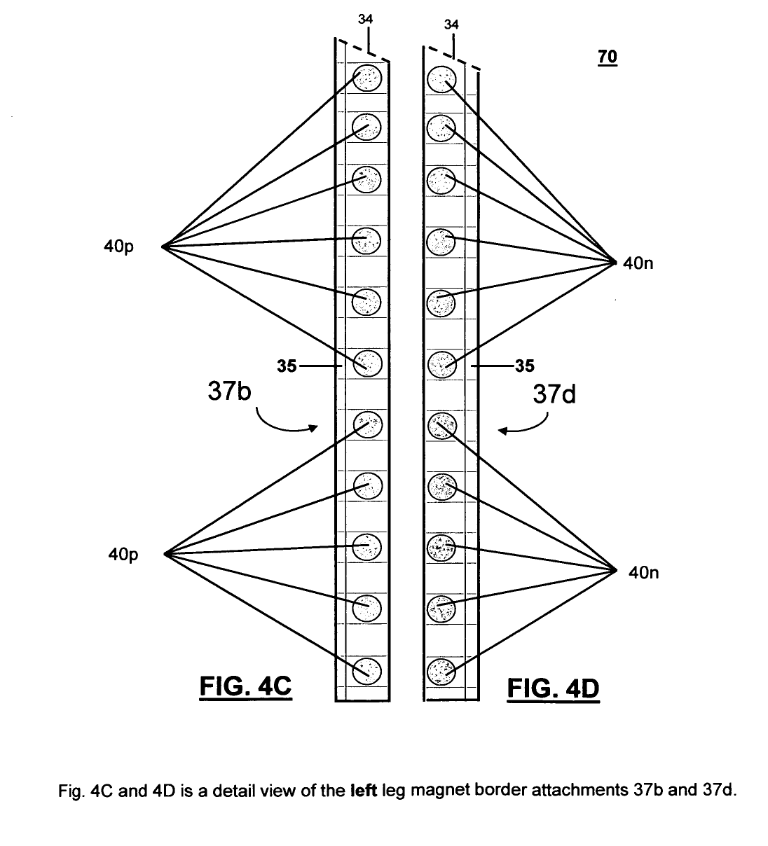

Referring to FIG. 4A through FIG. 4D are detail views of leg magnet border 36b, 36d, and 37b, 37d respectively. Each said leg magnet borders 36b, 36d, and 37b, 37d are made from the same material as the material of the massage drape sheet 1 and fabricated approximately 4½ inches in width and approximately 56 inches in length. The fabric is creased to reach a 2 inch width double layer by folding it over and an approximate ½ inch attachment sewing border 35 that will serve to attach the magnet borders to the massage drape sheet 1. The end of each border is cut and sewn to fit the leg/pelvic cut 32 of the massage drape sheet 1. About 11 magnets 40p in each leg magnet borders 36b and 37b are spaced approximately 3 to 4 inches apart from the top to the bottom in between the top and bottom layer of the fabric. Respectively, about 11 magnets 40n in each leg magnet border 36d and 37d are spaced approximately 3 to 4 inches apart from the top to the bottom in between the top and bottom layer of the fabric. Each individual magnet located inside the leg magnet borders 36b, 36d, and 37b, 37d respectively will then be sewn with a stitching line 41 on each side to create a chamber which keeps the magnet in place, in proper magnetic force position, and from moving within the border container. The entire border is then sewn elongated together to close the border. Each respective leg magnet border 36b, 36d and 37b, 37d is then attached to the pelvic magnet border 36a, 36c and 37a, 37c at the sewing line 34 and as illustrated in FIG. 5 before being attached for each right/left; leg/pelvic cutting edge to the massage drape sheet 1.

Referring to FIG. 5 is an exploded detail view referencing FIG. 3A/FIG. 3B and FIG. 4A/FIG. 4B as a unit. The right pelvic magnet borders 36a and 36c illustrate the connection of said right pelvic magnet borders to the right leg magnet borders 36b and 36d at the connecting sewing line 34. Accordingly, the pelvic and leg borders for the left side are connected in the same manner (position not shown).

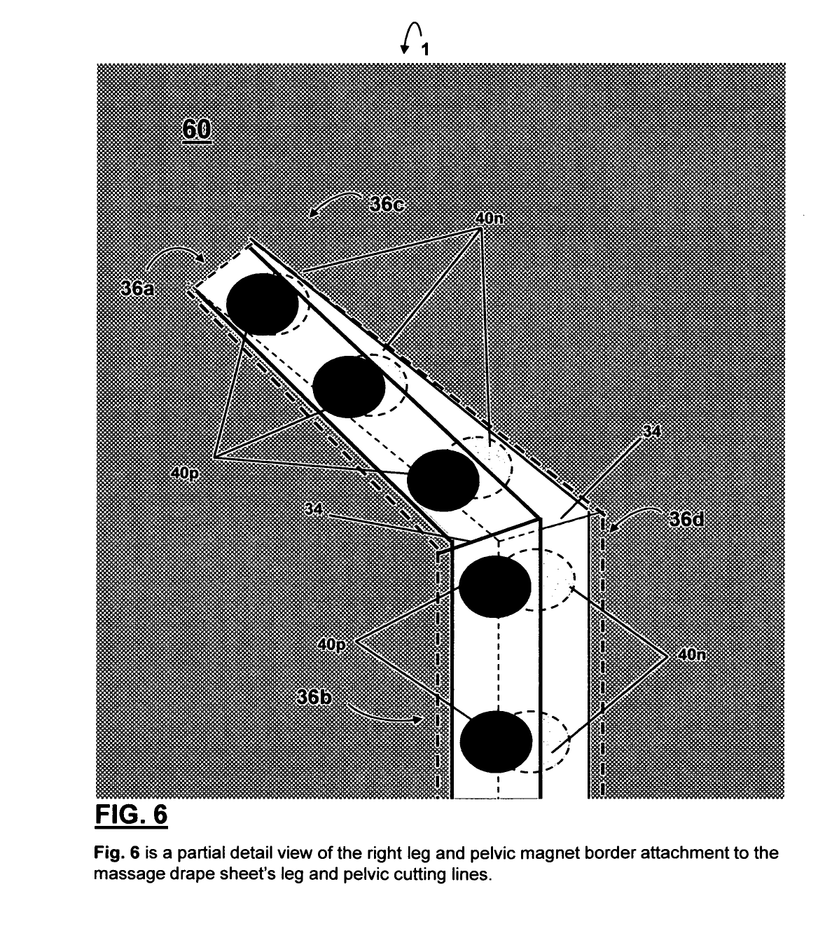

Referring to FIG. 6 is another detail view of the pelvic/leg magnet attachment border to the right pelvic/leg opening cutting lines 32 attaching the border to the massage drape sheet 1 of the present invention. The pelvic/leg magnet borders 36a and 36b (as one unit) are attached to the inner cutting edge of the right leg opening cut 32. The pelvic/leg border is then sewn from the top starting point of the pelvic angle cut along the inner cutting edge using the excess approximate ½ inch border to attach the border to the massage drape sheet 1 along side the leg cut continued elongated to the bottom edge of the massage drape sheet's 1 border.

The pelvic/leg magnet borders 36c and 36d (as one unit) are attached to the outer cutting edge of the right leg opening cut 32. The pelvic/leg border is then sewn from the top starting point of the pelvic angle placing and sewing the pelvic magnet border 36c directly flush over the pelvic magnet border 36a and then continue sewing along the outer cutting edge using the excess approximate ½ inch border to attach the border to the massage drape sheet 1 elongated the leg cut to the bottom edge of the massage drape sheet's 1 border. Accordingly, the pelvic and leg borders for the left side are connected in the same manner (position not shown).

Referring to FIG. 7 is a detail view of the fabric patch 42 made from the same material as the massage drape sheet 1 and is about 1 inch in width and 1 inch in length. The magnet 40n is placed with the negative magnetic side up and then sewn in a square as indicated on line 43 onto the massage drape sheet as illustrated in FIG. 2.

Referring to FIG. 8 is a partial detail view of the abdomen veil 23 illustrating the placement of the magnets 40p with the positive magnetic side up. These magnets are sewn inside the veil's approximate 1 inch border. Each magnet is separated by a right and left side stitched line 41 to create a chamber and keep the magnets in place.

### DETAILED DESCRIPTION

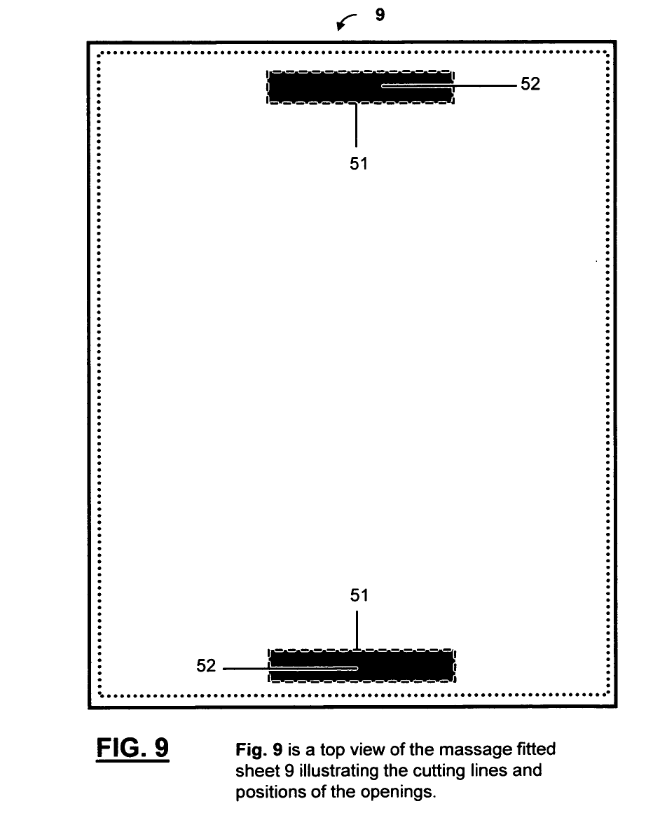

### FIG. 9—Preferred Embodiment

Is a full detail of a second separate massage fitted sheet 9 made from the same material as the massage drape sheet 1. The massage fitted sheet 9 is approximately 35 inches in width; approximately 77 inches in length; and approximately 7.5 inches in depth created and sewn as the conventional fitted bed sheets. The preferred embodiments of the current invention of the massage fitted sheet 9 is one opening located at each end of the sheet to serve as access points for the massage table's foot and/or head rest devices. Each opening is located about 3 inches from the sheet's border edge and approximately 18.5 inches from the left and the right side respectively. The length of the opening is approximately 11 inches and approximately 1 inch in width with a ½ inch hem border around the opening.

### ADVANTAGES

From the description above and the drawings attached some very important aspects of my massage drape invention become evident. Although historically the profession of massage therapy has used bed sheets for many years the issues with legal draping requirements are rising because of the sheets inability to accommodate such requirements. My invention is the result of such exposure and inventing a sheet that is more a tool to the therapist but yet maintaining the core comfort of coverage to the client. Bed sheets are made from one solid piece of fabric where my invention includes an opening to access the stomach yet is covered with a veil when the opening is not needed. It also includes a right and left leg opening that reaches all the way to the pelvic region yet can be closed at the seam line when not needed. It represents a one piece sheet such as the sheet a massage therapist is used to but a tool of accessibility to body parts when generic one piece bed sheets fail. A one piece bed sheet or even one that is folded in half still has to be lifted at the lateral side in its entirety to reach the leg. This lifting is a tremendous opportunity for lateral exposure. My invention solved this problem by creating three moveable pieces which remain on either side of the legs and a middle piece in the crutch area. Additionally, law now states that no nipples are to be exposed during a massage which means male and female clients are affected. Today a massage therapist has to use a second piece of fabric which is a pillow case in most instances to first place the cover over the drape sheet than carefully remove the drape sheet from underneath while the client is holding the pillow case in place over the breast or breast tissue area. This is a lot of interruption and again a tremendous amount of exposure opportunity. My invention has resolved this issue with a no more than two second solution by cutting an opening covered with a veil directly into the sheet that allows for the exposure of the needed stomach area.

Also, the closure mechanism used with magnets is reflective of the nature of this industry which requires these three major components: being quiet, being fast, and being secure. The magnet borders containing the magnets allow for a fastening mechanism that is easy to separate and re-close the openings needed to instantaneously access the body parts. This in return allows the therapist to maintain an uninterrupted flow of the massage.

These features from my invention will elevate a higher level of comfort for the clients and reduces time for the therapists. As the magnets in this present invention are used for the open and closing mechanism, they also serve additional purposes supporting the efforts of massage therapy. Magnets are known to theorize therapeutic healing powers. The stationary magnets embedded in the massage drape 1 are joined together with the therapist's professional service and a metal widely accepted for such healing powers thus creating a unique added value therapeutic device and tool combined as one. Prior to the preferred embodiment of the current invention, magnets and massage therapy modalities are generally two separate service components because you can't place magnets directly onto a client's skin. The embodiment of the current invention includes the magnets inside the massage drape sheet 1, therefore utilizing the magnetic fields while receiving a massage.

The second sheet of the massage fitted sheet is 9 a modification of the fitted bed sheet design adding two openings on each end and adjusting the size to fit most all professional massage tables. The openings allow for the head and foot rest attachment device to easily slip into the table without having to hold or handle the fitted sheet prior to attaching the device. Because of the major modification made by my current invention of the massage drape sheet 1 it is only logical to have the fitted sheet also modified in such a way that it fits with the need and design of a massage table. This added invention of the fitted sheet 9 combined with the invention of the massage drape sheet 1 create the massage draping sheet set.

### CONCLUSION, RAMIFICATIONS, AND SCOPE

In conclusion to my present invention, the massage drape sheet 1 as well as the massage fitted sheet 9 represents professional tools for the massage industry and/or any profession where exposure of necessary body parts yet coverage of the remaining body are required. Bed sheets are made for beds and my invention is made specifically for these professions. Preliminary manufacturing processes have shown that my invented sheets are producible in a cost effective and efficient way.

Although, the current invention of the massage drape sheet 1 and massage fitted sheet 9 are described with reference to the preferred embodiments and with approximately specific measurements, it will be understood and should not be construed that various obvious changes may be made, and equivalents may be substituted for elements thereof, without departing from the essential scope of the present invention. Therefore, the scope of the invention should be determined by the appended claims and their legal equivalents, rather than by the examples given in this document. For example, the magnets can be square shaped or made from other magnetic materials that create the same magnetic force for a closure mechanism. Other closure systems may be employed without deviating from the core elements of the present invention. Other cuts to create openings within the sheet may be introduced that create the accessibility to body parts while maintaining overall coverage.

What I claim is:

1. A massage drape sheet being of rectangular shape and textile material having openings for means to access particular human body parts being massaged while maintaining draping coverage. Wherein said massage drape sheet having a part or parts of the said sheet material removed through cutting that part out creating said opening.
Wherein said opening having a veil like cover made from a separate piece of textile being of rectangular shape attached to the said massage drape sheet as means to cover the opening. Wherein said veil cover's border employs a fastening system for open and closing said veil.
Wherein said massage drape sheet also has a plurality of partial elongated and angled slits that are cut into the said massage drape sheet separating the textile woven parts creating an opening as means to access anatomical body parts. Wherein said slits employ attachment sites as means for attaching a fastening system.
Wherein said massage drape sheet is an article of sheet like material to cover a nude human body while being massaged, comprising of:
a) a veil covered opening with a fastening system
b) a plurality of slit like openings with a fastening system

2. The massage draping sheet of claim 1 wherein the sheet is made from natural fibers or a blend thereof.

3. The massage draping sheet of claim 1 has a permanent opening of a length and width to expose the region of the human abdominal body part.

4. The massage draping sheet of claim 1 that has a veil of rectangular shape that has a length and width to cover the said opening entirely and is made from cotton textile material or a blend thereof.

5. The massage draping sheet of claim 4 of said veil having a fastening system that has a plurality of magnets inside the said veil's border positioned to attract to opposite magnets.

6. The massage draping sheet of claim 4 where said opposite magnets are positioned and permanently attached opposite onto said massage draping sheet with a patch of fabric.

7. The massage draping sheet of claim 1 has a right and left leg opening slit of a length and angle to the pelvic region to expose the region of the human leg and pelvic body part.

8. The massage draping sheet of claim 7 wherein the said slit employs an inner and outer attachment site for means of attaching a plurality of magnet borders.

9. The massage draping sheet of claim 7 wherein said magnet borders contain the magnets for the closing and opening mechanism.

10. The massage draping sheet of claim 1 having a fastening system in shape of a border containing a plurality of magnets to close and open said openings and slits.

11. The massage draping sheet of claim 10 of said fastening system having a plurality of opposite poled magnet pairs positioned in direct opposite location from each other.

12. A massage fitted sheet being of rectangular shape made from textile material having a length and width that covers professional sized massage tables. Wherein said massage fitted sheet having a part or parts of the said sheet material removed through cutting that part out creating an opening.

13. The massage fitted sheet of claim 12 having one opening at each end of the said sheet.

14. The massage fitted sheet of claim 12 wherein the sheet is made from natural fibers or a blend thereof.

15. A massage draping sheet set comprised of a massage drape sheet being of rectangular shape and textile material having openings for means to access particular human body parts being massaged while maintaining draping coverage. Wherein said massage drape sheet having a part or parts of the said sheet material removed through cutting that part out creating said opening.
Wherein said opening having a veil like cover made from a separate piece of textile being of rectangular shape attached to the said massage drape sheet as means to cover the opening. Wherein said veil cover's border employs a fastening system for open and closing said veil.
Wherein said massage drape sheet also has a plurality of partial elongated and angled slits that are cut into the said massage drape sheet separating the textile woven parts creating an opening as means to access anatomical body parts. Wherein said slits employ attachment sites as means for attaching a fastening system.
Wherein said massage drape sheet is an article of sheet like material to cover a nude human body while being massaged, comprising of:
c) a veil covered opening with a fastening system
d) a plurality of slit like openings with a fastening system
And a massage fitted sheet being of rectangular shape made from textile material having a length and width that fits professional sized massage tables. Wherein said massage fitted sheet having a part or parts of the said sheet material removed through cutting that part out creating an opening.

16. A professional massage draping sheet set of claim 15 having a said massage drape sheet.

17. A professional massage draping sheet set of claim 15 having a said massage fitted sheet.
